(19) United States
(12) Patent Application Publication
(10) Pub. No.: US 2008/0257357 A1
(43) Pub. Date: Oct. 23, 2008

(54) Dental laryngeal mask

(76) Inventors: Allan Schwartz, Columbia, MO (US); Sanjay H. Parikh, Finksburg, MD (US)

Correspondence Address: Paul Grandinetti;LEVY & GRANDINETTI, P.O. Box 18385, Washington, DC, 20036, US

(21) Appl. No.: 12/078,324

(22) Filed: Mar. 28, 2008

Related U.S. Application Data
(60) Provisional application No. 60/907,354, filed on Mar. 29, 2007.

(51) Int. Cl. A61M 16/00

(52) U.S. Cl. 128/207.15

(57) ABSTRACT

The invention is a laryngeal mask and method for using such a mask. The laryngeal mask includes a dome, a cuff, and an air tube or passageway. The tube of the laryngeal mask device is capable of being temporarily folded, collapsed, or disconnected so that the portion of the tube passing between the teeth is repositioned to permit the teeth to close temporarily.

We claim the benefit under Title 35, United States Code, §119 of U.S. Provisional Application No. 60/907,354, filed Mar. 29, 2007, entitled DENTAL LARYNGEAL MASK.

### BACKGROUND OF THE INVENTION

1. Field of the Invention

This invention relates to a dental laryngeal mask and method for using such a mask. Specifically, this invention relates to a laryngeal mask with the ability to be temporarily disconnect at least one portion of the tube so that a dental professional can check the occlusion of the teeth while the remainder of the laryngeal mask device is seated in its proper place above the glottic opening in the hypopharynx.

2. Description of Related Art

Laryngeal masks are well known and are commonly used by medical, emergency medical, dental, and veterinary professionals to intubate an unconscious patient for anaesthetic and ventilation purposes. A laryngeal mask is inserted into the throat or pharynx of an unconscious patient, such as an accident victim, a dental patient. The laryngeal mask forms a seal around the laryngeal inlet and prevents the epiglottis from blocking the lumen. The laryngeal mask provides an open airway to permit the unconscious patient to breathe and be provided anesthesia.

Laryngeal masks are made by numerous manufacturers and have similar structures for the basic components. These manufacturers use standard terminology for the common components of these devices.

Typically, the basic components of a laryngeal mask include a dome, an airway tube or shaft, and a cuff or rim. The dome is a teardrop-shaped, semi-rigid member wherein the more angular portion of the teardrop shape provides the distal or leading edge for insertion into a patient's larynx. A rigid, yet flexible, tube is inserted into an orifice in the proximal portion of the dome and projects from this rounded portion of the dome. The flexible tube is of sufficient length to traverse a curve or angle from the larynx of a patient to a point outward from the patient's mouth. An inflatable cuff encircles the periphery of the dome and is made of soft, flexible material. When the dome is positioned in the larynx of the patient, the cuff is inflated and forms a seal with the surrounding tissue in the upper esophageal sphincter, pyriform fossae, and esophageal inlet to secure the laryngeal mask in place and to ensure that the internal lumen of the mask is positioned over the laryngeal inlet. Various sizes of laryngeal airway devices are available to accommodate different sizes of patients which include children, adults, and animals. It is desirable that the laryngeal mask be constructed of medical-grade materials that can withstand repeated autoclaving to allow repeated use of the laryngeal mask device. Lesser medical-grade materials can be used for single-use or “disposable” laryngeal mask devices.

The industry strives to improve these individual components as well as the collateral components used in these devices. Collateral components include inflation tubes for the cuff, devices for inserting the laryngeal mask without damaging nerve or other structures, and similar components.

The industry lacks a sanitary laryngeal mask and a method for using the laryngeal mask for dental rehabilitation under general anesthesia. Such a laryngeal mask must permit the occlusion or “bite” of the teeth be checked with the laryngeal mask operating in the patient's throat so as to permit continued respiration.

### SUMMARY OF THE INVENTION

The invention includes a laryngeal mask having a dome and an airway tube extending from a proximal passage through the dome. The tube of the laryngeal mask device can be temporarily folded, collapsed, or disconnected so that the portion of the tube passing between the teeth is repositioned to permit the teeth to close temporarily.

Desirable embodiments of the invention easily allow the tube of the laryngeal mask apparatus to temporarily disconnect and then reconnect while providing a sanitary, air-tight seal to prevent water, blood, and debris, associated with dental procedures, from entering the patient's airways. The tube can temporarily disconnect to allow the dental professional to check the occlusion or “bite” of the teeth with the lower portion of the dental laryngeal mask apparatus remains properly positioned within the patient.

The tube can be moved from between the teeth or otherwise disconnected and reconnected in a variety of ways. For example, a tube joint can be formed by parts that (1) screw and unscrew together, (2) attach by a friction fitting, (3) slide, turn, and lock together, (4) twist and lock together, (5) or provide an accordion-type folding for shortening the tubing into the patient's mouth, or (6) other structures that permit the gentle removal from between the teeth with a secure repositioning to maintain an open airway.

### BRIEF DESCRIPTION OF THE DRAWINGS

FIG. 1 is a perspective view of the laryngeal mask of the invention showing the tube temporarily disconnected with a friction-fit divided tube joint.

FIG. 2 is a perspective view of the laryngeal mask of the invention showing the tube temporarily disconnected with a slide, turn, and lock tube joint.

FIG. 3 is a perspective view of the laryngeal mask of the invention showing the tube temporarily disconnected with a screw-fit tube joint.

FIG. 4 is a perspective view of the laryngeal mask of the invention showing the tube temporarily disconnected with a accordion-type tube joint.

### DETAILED DESCRIPTION OF THE INVENTION

The invention is a laryngeal mask and method for using such a mask. The laryngeal mask includes a dome, a cuff, and an air tube or passageway. The tube of the laryngeal mask device is capable of being temporarily folded, collapsed, or disconnected so that the portion of the tube passing between the teeth is repositioned to permit the teeth to close temporarily.

A desirable embodiments of the invention easily permit the tube of the laryngeal mask device to temporarily disconnect by providing a joint in the tube that is friction fitted, screwed, or snapped together. When the joint is fully screwed together or otherwise reattached, it provides an air-tight seal for the extended tube. When the tube of the laryngeal mask device is folded, collapsed, or unscrewed, is allows the medical professional an opportunity to close the mouth of the patient for a variety of reasons including to check the occlusion or “bite” of the teeth with the lower portion of the dental laryngeal mask apparatus remaining properly positioned within the patient.

Another desirable embodiment of the invention permits the tube of the laryngeal mask device to temporarily disconnect by providing a joint in the tube that fits together with a friction fitting. The tube separates into two sections. The joint consists of an upper section and a lower section. The upper section of the joint slides over and overlaps a portion of the lower section of the joint. The inside rim of the upper section of the joint and the outside rim of the lower section of the joint are constructed of a rough texture and/or with adhering surfaces so that friction or adherence is produced when they are slid against one another to provide a secure seal in the tube of the laryngeal mask device.

Another desirable embodiment of the invention permits the tube of the laryngeal mask device to temporarily disconnect by providing a joint in the tube that fits together with a slide-turn-and-lock connection. A first end of the joint has a raised member. A second end of the joint has a groove cut into the joint in a configuration capable of fitting or complementing the raised member of the first end of the tube. The groove of the second end can be changed to a second direction after a distance so that when the first end of the tube with the raise member is twisted to the second direction the joint locks the raised member of the first end into position and provide a secure seal in the tube of the laryngeal mask device.

A further desirable embodiment of the invention permits the tube of the laryngeal mask device to remain one solid piece but with an accordion-style or comparable folding of the tubing. A collapsing or folding structure permit the compression of the tubing into the patient's mouth. The shortened tubing permits the tube to retain a solid seal while also allowing a medical professional an opportunity to close the mouth of the patient for a variety of reasons including to check the occlusion or “bite” of the teeth while the lower portion of the dental laryngeal mask apparatus remains properly positioned within the patient.

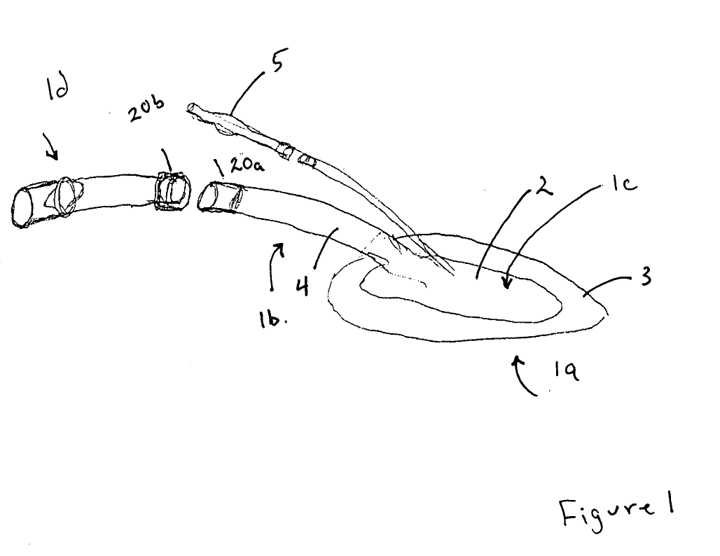

FIG. 1 illustrates a laryngeal mask 1 according to the invention having a distal end 1a, proximal end 1b, posterior portion 1c, and anterior portion 1d. The laryngeal mask includes a dome 2 having a cuff 3. An airway tube 4 is tightly secured to the posterior proximal portion of the dome 2. An inflation means 5 is attached to the proximal end of the cuff 3 and is adapted for connecting with a pump or inflation (not shown). This embodiment uses a friction or adherence fit to removably attach the male connection 20a into the female connection 20b.

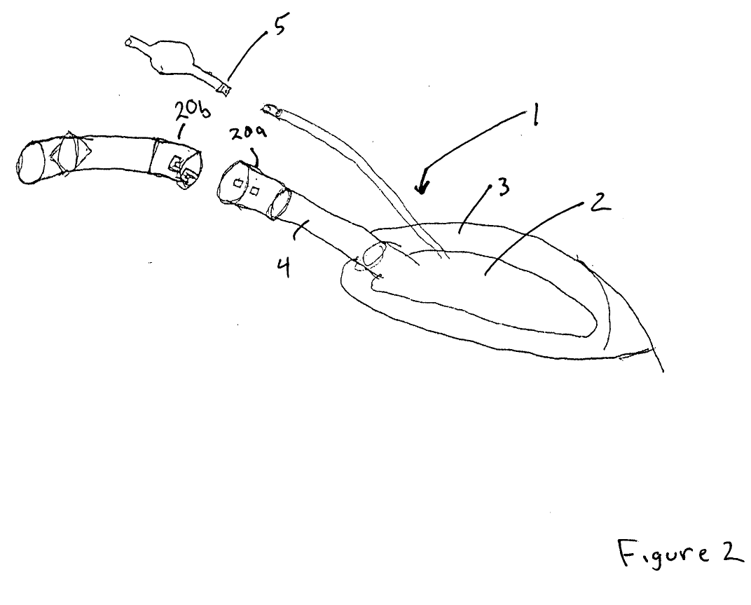

FIG. 2 illustrates a anterior view of the laryngeal mask 1. This view of the invention demonstrates the passage through the dome 2 into the airway tube 4. The attachment of the airway tube 4 to the dome 2 must be secure such that no separation is possible when the laryngeal mask is being removed from a patient. This figure also illustrates the preferred attachment of the inflation means 5 to the proximal end of the cuff 3. This figure illustrates the cuff 3 in an inflated condition. This embodiment uses a turn and lock connection to removably attach the male connection 20a into the female connection 20b.

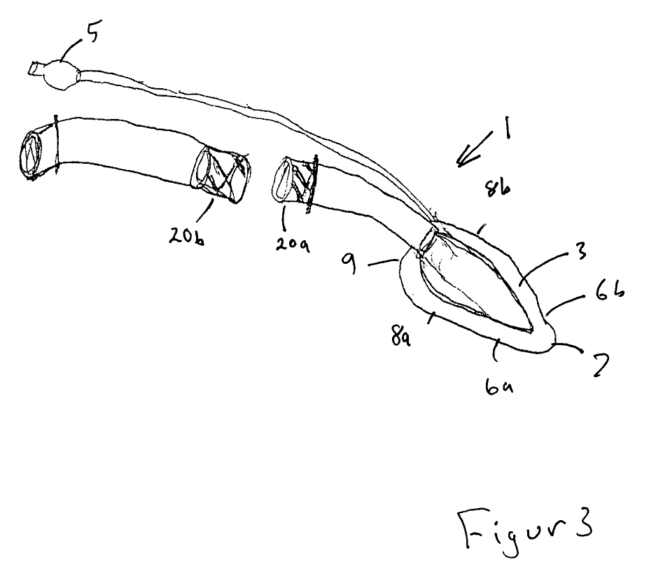

FIG. 3 illustrates the laryngeal mask 1 with the cuff 3 in a deflated condition. The cuff 3 is deflated to reduce its overall size and to form a shape which facilitates the insertion of the laryngeal mask 1 into the hypopharynx of a patient. It is desirable for the cuff 3 to deflate uniformly as well as consistently after repeated uses. A desirably deflated cuff 3 collapses in complementary distal portions 6a and 6b. The collapse of the cuff 3 in complementary distal portions 6a and 6b forms an angular shape to facilitate insertion into a patient's upper esophageal opening. It is desirable for the distal tip 7 to be firm enough to separate the epiglottis from the posterior hypopharynx. However, it is also important that the distal tip 7 be sufficiently soft so as not to damage these physiological structures or the lingual nerve. This embodiment uses a screw-fit to removably attach the male connection 20a into the female connection 20b.

The deflated cuff 3 also deflates in complementary lateral portions 8a and 8b. The collapse of the cuff 3 on its lateral portions reduces the cross section of the laryngeal mask being inserted into a patient's esophagus. The cross section (not shown) of the collapsed cuff 3 and dome 2 of the laryngeal mask 1 desirably does not exceed the internal diameter of the patient's esophagus. It is common for laryngeal masks, including those according to the invention, to be manufactured in a variety of sizes because of variations in the internal diameters of patients' esophagi. However, careful selection of the dimensions of the dome 2 and cuff 3 can provide a universal laryngeal mask for most adult patients. For infants, manufacturers typically provide pediatric-sized laryngeal masks.

The deflated cuff 3 also collapses at distal portion 9. Distal portion 9 is desirably the location where the inflation means 5 securely connects to the cuff 3. Other embodiments can include a filled or “expanded” cuff. A filled cuff can be fully or partially filled with a foam, suspension, or other material. When a fill cuff is partially filled with a material, it can be fully inflated with air.

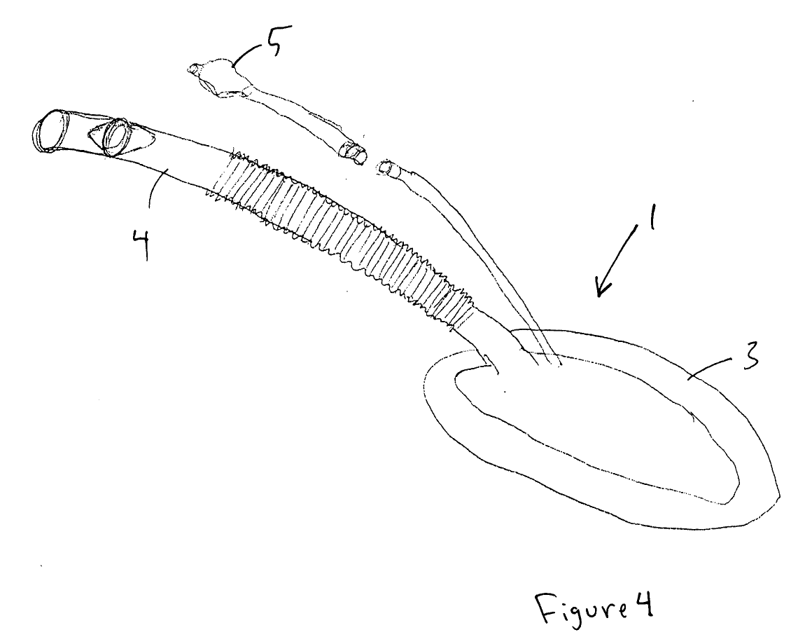

FIG. 4 illustrates an embodiment wherein the tube 4 uses an accordion structure to permit the tube 4 to be collapsed into a patient's mouth. The tube 4 remains a solid piece.

Laryngeal masks according to the invention are desirably made from one or more polymeric materials. The materials must permit adhering or fusing, for example, of the tube to the dome and the cuff to the dome. Complimentary polymeric materials can be soft polymers such as latex, polyurethane, or similar natural or synthetic resin material. Polymeric materials can provide frictional, adhering services for the divide tube or hard surfaces that permit easy sliding or screwing of the joint portions of the divided tube.

The invention includes a method for using a laryngeal mask. The method includes providing a laryngeal mask with a divided tube or tube that collapses or folds. The laryngeal mask with the tube section joined together is inserted into a patient's esophagus. When inspection of the patient's bite is performed, the tube is separated or it is collapsed or folded into the patient's mouth. The inspection is performed and the tube is reattached or extended permitting the patient to breath freely. The laryngeal mask is removed after the procedure.

The benefit of this invention is that it provides a simple, reliable structure that is permits temporarily disconnection or shortening of the tube so that a dental professional can check the occlusion of the teeth while the remainder of the laryngeal mask device is seated in its proper place above the glottic opening in the hypopharynx.

We claim:

1. A laryngeal mask comprising:
a dome;
an airway tube extending from a proximal passage through said dome, said tube has at least two sections that removably attach while said laryngeal mask in inserted into a patient; and
a cuff of flexible material, said flexible material has a first longitudinal edge and a second longitudinal edge, said first longitudinal edge and said second longitudinal edge are joined to form a tube for said cuff, said cuff substantially encircles said periphery of said dome.

2. A laryngeal mask comprising:
a dome;
an airway tube extending from a proximal passage through said dome, said tube reversibly collapses or folds while said laryngeal mask in inserted into a patient; and
a cuff of flexible material, said flexible material has a first longitudinal edge and a second longitudinal edge, said first longitudinal edge and said second longitudinal edge are joined to form a tube for said cuff, said cuff substantially encircles said periphery of said dome.
